(19) United States
(12) Patent Application Publication
(10) Pub. No.: US 2023/0407353 A1
(43) Pub. Date: Dec. 21, 2023

(54) CELL-FREE METHODS OF PRODUCING PIGMENTS

(71) Applicant: DEBUT BIOTECHNOLOGY, INC., San Diego, CA (US)

(72) Inventors: Joshua Britton, San Diego, CA (US); Jeff Ehrhardt, San Diego, CA (US)

(21) Appl. No.: 18/210,193

(22) Filed: Jun. 15, 2023

Related U.S. Application Data
(60) Provisional application No. 63/352,761, filed on Jun. 16, 2022.

(51) Int. Cl. C12P 17/12

(52) U.S. Cl. CPC C12P 17/12

(57) ABSTRACT

The invention is directed to methods of enzymatic production of betalains, including betalamic acid, in cell-free medium.

### I. FIELD OF INVENTION

The invention is related to materials and methods for production of pigments. The pigments of the inventions are useful as color additives in cosmetics, nutrition, food, and beverage applications.

### II. BACKGROUND OF THE INVENTION

Betalains are tyrosine-derived, red-violet and yellow plant pigments found primarily in one group of angiosperms, the Caryophyllales order, in which they occur in a mutually exclusive fashion with the chemically distinct and widespread anthocyanin pigments. The betalain class contains a wide array of compounds, which are generally classified into two groups; the red-violet betacyanins, and the yellow betaxanthins.

Betalains are commonly used as coloring agents in cosmetics, nutrition, food, and beverage products. Betalains are nitrogen-containing pigments responsible for the coloration of plants of the order Caryophyllales. Betalains are classified into two groups: betaxanthins and betacyanins. Both groups have betalamic acid as a chromophoric and structural unit and condense with different molecules. Betaxanthins are derived from the condensation of betalamic acid with amines and amino acids. These compounds have a maximum absorbance spectrum centered on a 480 nm wavelength and have a yellow coloration. Betacyanins are the result of the condensation of betalamic acid with indoline-type cycled amines.

Due to their high temperature stability, pH stability, and antioxidative properties, betalains may be used as natural food colorants, cosmetic pigments, and dietary supplements. Color changes in pigments are usually related to chemical modification, which could potentially compromise health benefits and safety. Betalains are highly stable colors and Betalains exhibit a broad pH stability adequate for low-acidity foods. Betaxanthins are most stable at pH values ranging from 5.5 to 7, whereas betacyanins are considered to exhibit optimum stability in a narrower pH range (i.e., between 5 and 6) and betalamic acid remains intact at pH 9. Betalains are also used as antioxidants.

There have been previously known processes for manufacturing betalains. Sources of betalains used for food-coloring purposes contain, among other substances, a mixture of betanin and its epimer, isobetanin.

### III. SUMMARY OF THE INVENTION

The chemical synthesis of betalains does not seem feasible for the commercial production of pigments due to the numerous steps involved in betalamic acid synthesis and the low yield of the final products. Therefore, the commercial success of betalain production depends on both cost and the availability of source plant materials, as well as on the extraction efficiency. In particular, obtaining betalains and purifying them from whole plants have depended directly on availability throughout the year and on environmental variables that affect their production. Moreover, losses during processing of plant material due to low pigment stability jeopardize the commercial extraction of betalains. Further, flavor compounds such as geosmin are challenging to remove from plant-extracted betalains and add undesirable flavor profiles to plant-extracted betalains.

The invention provides a feasible route for the rapid, safe, economical, and sustainable production of betalains, including betalamic acid. It is an objective of the present invention to provide a cell-free method that allows for the production of betalains.

In the cell-free systems described herein, the critical components of the cell, namely cofactors and enzymes, are used in a chemical reaction without cellular components that can directly or indirectly inhibit the desired biochemical reaction. The same enzymes found in plants and other organisms may be created in vivo (typically through protein overexpression in hosts such as bacteria), isolated via chromatography, and then added into a bioreactor with a substrate (starting material). The enzymes transform the substrate in the same way that occurs in the original organism without the organism's complexity. Additionally, the biochemical reaction may be enhanced by the addition of co-solvents, detergents, or both, which would not be tolerated by, or simply would not work in a whole cell-based manufacturing method. In this way, natural products can be created without the plant, cell, or chemical synthesis.

A unique and inventive technical feature of the present invention is the use of a cell-free system for the production of a large number betalains. The invention advantageously provides for higher reaction concentrations of one or more of starting materials, reagents, and/or enzymes, resulting in higher concentrations of final products (e.g., betalains or betalamic acid). Additionally, the present invention eliminates the complications of cell walls, thereby eliminating a significant barrier to product and substrate diffusion. Furthermore, the present invention eliminates competition for carbon flux, which limits the efficiency of cell-based synthesis methods, and thus greatly reduces byproduct formation. Also, because there is no cell, the present invention is not vulnerable to degradation by cell death due to the formation of toxic compounds. In addition, the present methods enhance the ability to use various solvents, such as organic solvents, to permit higher concentrations of solutes (e.g., substrates and intermediates) without worrying about killing the cell. Certain embodiments of this invention further use immobilized enzyme(s) for downstream processing. The use of immobilized enzyme is advantageous because it simplifies downstream processing of products, purification of products, and creating easier access to high purity pigments. The invention also provides for cell-free enzyme processes that allow enzyme(s) to be recycled. Such enzymes of the invention could be preferably recycled through downstream processing units such as filtration and centrifugation.

Aspects of the invention provide a method of cell-free production of a betalain, wherein one or more enzymes result in transformation of an organic material to a betalain. The invention further provides that in certain aspects the betalain is betalamic acid. In certain embodiments of the invention, the organic material is 3,4-dihydroxyphenylalanine (DOPA). In certain embodiments of the invention, the organic material is 1-3,4-dihydroxyphenylalanine (L-DOPA). In certain embodiments, the one or more enzymes resulting in the transformation of organic material to betalamic acid is an engineered enzyme. In certain embodiments of the invention, 3,4-dihydroxyphenylalanine 4,5-dioxygenase (DODA) transforms DOPA or L-DOPA to betalamic acid. In certain embodiments, DODA could be an engineered enzyme. In certain embodiments of the invention, transformation proceeds through one or more chemical intermediates and the one or more chemical intermediates. In certain embodiments of the invention, one or more chemical intermediates comprises 4-(L-alanin-3-yl)-2-hydroxy-cis,cis-muconate 6-semialdehyde (4,5-seco-DOPA). In certain embodiments of the invention, betalamic acid is further reacted with one or more amino acids and/or amines to prepare one or more pigments. In certain embodiments of the invention, the one or more pigments are colors for addition to food. In certain embodiments of the invention, the one or more pigments is betalamic acid, betanin, betanidin, betacyanin, betaxanthin, betalain, or a combination thereof. In another embodiment, the enzyme resulting in transformation of the organic material to a betalain is immobilized on a surface. In another embodiment, the enzyme resulting in transformation of the organic material to a betalain is not immobilized.

In other aspects of the invention, a composition for cell-free production of a betalain is provided. In such aspects of the invention, the composition comprises an enzyme that results in transformation of an organic material to a betalain. In certain aspects, the invention further provides that the betalain is betalamic acid. In certain embodiments of the invention, the organic material is any stereoisomer of 3,4-dihydroxyphenylalanine DOPA. In certain embodiments of the invention, the organic material is 3,4-dihydroxyphenylalanine (DOPA). In certain embodiments of the invention, the organic material is 1-3,4-dihydroxyphenylalanine (L-DOPA). In certain embodiments, the one or more enzymes resulting in the transformation of organic material to betalamic acid is an engineered enzyme. In certain embodiments of the invention, 3,4-dihydroxyphenylalanine 4,5-dioxygenase (DODA) transforms DOPA or L-DOPA to betalamic acid. In certain embodiments, DODA could be an engineered enzyme. In certain embodiments of the invention, transformation proceeds through one or more chemical intermediates and the one or more chemical intermediates. In certain embodiments of the invention, one or more chemical intermediates comprises 4-(L-alanin-3-yl)-2-hydroxy-cis,cis-muconate 6-semialdehyde (4,5-seco-DOPA). In certain embodiments of the invention, betalamic acid is further reacted with one or more amino acids and/or amines to prepare one or more pigments. In certain embodiments of the invention, the one or more pigments are colors for addition to food. In certain embodiments of the invention, the one or more pigments are colors for addition to cosmetics. In certain embodiments of the invention, the one or more pigments are ingredients for nutraceutical applications. In certain embodiments of the invention, the one or more pigments is betanin, betanidin, betacyanin, betaxanthin, betalain, or a combination thereof. In another embodiment, the enzyme resulting in transformation of the organic material to a betalain is immobilized on a surface. In another embodiment, the enzyme resulting in transformation of the organic material to a betalain is not immobilized. In another embodiment, betalamic acid is immobilized on a resin surface during production.

### IV. BRIEF DESCRIPTION OF THE DRAWINGS

FIG. 1 shows that DOPA is converted into betalamic acid (center), which can be derivatized into yellow betaxanthins (left) and variable colored betacyanins (righ.

FIG. 2 shows the biosynthesis of betalamic acid and downstream derivatization to betalains. A representative set of betalains and the amines used to form them are also provided.

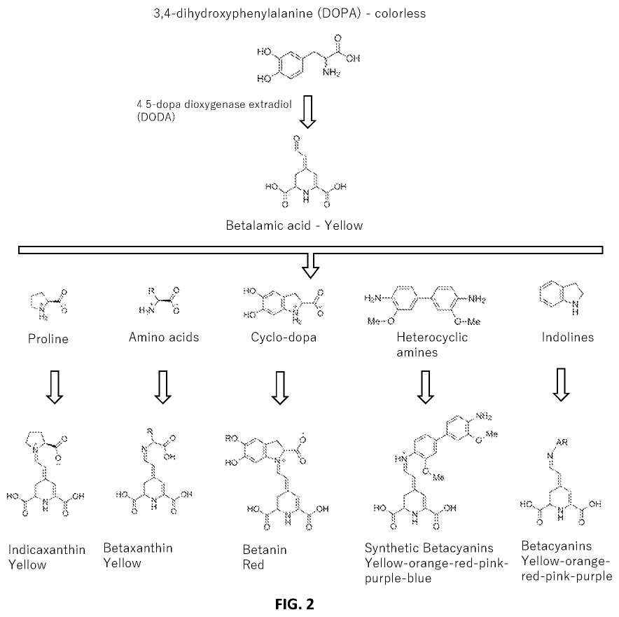

FIG. 3 shows cell-free synthetic scheme to generate betalains. DOPA is cyclized into betalamic acid, which is purified or converted in situ to betalains by reaction with other amines or amino acids.

### V. DETAILED DESCRIPTION OF THE INVENTION

The present application provides compositions and methods for production of betalains in a cell-free medium, wherein one or more enzymes in a cell-free medium, wherein the one or more enzymes result in transformation of an organic material to a betalain. The one or more enzymes may be engineered. The engineered enzyme may be non-naturally occurring.

The term “non-naturally occurring”, when used in reference to an enzyme is intended to mean that nucleic acids or polypeptides include at least one genetic alteration not normally found in a naturally occurring polypeptide or nucleic acid sequence. Naturally occurring nucleic acids, and polypeptides can be referred to as “wild-type” or “original”. A host cell, organism, or microorganism that includes at least one genetic modification generated by human intervention can also be referred to as “non-naturally occurring”, “engineered”, “genetically engineered,” or “recombinant”.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, “reaction solution” may refer to all components necessary for enzyme-based chemical transformation. This is typically, but not limited to, buffering agent, salts, cofactor, and substrate (starting material).

As used herein, “DOPA” refers to all stereoisomers of 3,4-dihydroxyphenylalanine, including L-3,4-dihydroxyphenylalanine, D-3,4-dihydroxyphenylalanine, and mixtures of each stereoisomer.

As used herein, “L-DOPA refers to L-3,4-dihydroxyphenylalanine.

As used herein, “reaction mixture” may refer to all components from the “reaction solution” plus the enzyme(s) and/or products from the reaction. In some embodiments, the “reaction mixture” may refer to just the reaction solution without any enzymes or reaction products.

As used herein, “reaction mixture” may refer to all components from the “reaction solution” plus the enzyme(s) and/or products of the reaction. In some embodiments, “reaction solution” and “reaction mixture” may be used interchangeably.

As used herein, “buffering agents” may refer to chemicals added to water-based solutions that resist changes in pH by the action of acid-base conjugate components.

As used herein, “cofactors” may refer to a non-protein chemical compound that may bind to a protein and assist with a biological chemical reaction. Non-limiting examples of cofactors may include but are not limited to NADPH and NADH.

In the cell-free systems described herein, the critical components of the cell, namely cofactors and enzymes, are used in a chemical reaction without cellular components that can directly or indirectly inhibit the desired biochemical reaction. The same enzymes found in plants and other organisms may be created in vivo (typically through protein overexpression in hosts such as bacteria), isolated via chromatography and/or any other methods, and then added into a bioreactor with a substrate (starting material). The enzymes may also be used directly from plants without any isolation. The enzymes transform the substrate in the same way that occurs in the original organism without the organism's complexity. Additionally, the biochemical reaction may be enhanced by the addition of co-solvents, detergents, or both, which would not be tolerated by, or simply would not work in a whole cell-based manufacturing method. In this way, natural products can be created without the plant, cell, or chemical synthesis.

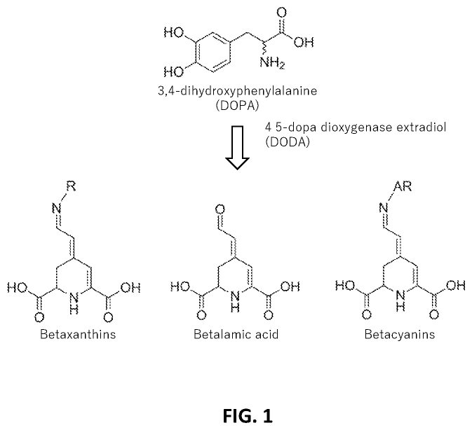

As shown in FIG. 1, the invention provides for a method of enzymatic transformation of 3,4-dihydroxyphenylalanine to betalains, including betacyanins, betalamic acid, and betaxanthins.

Aspects of the invention provide a method of cell-free production of a betalain, wherein one or more enzymes result in transformation of an organic material to a betalain. The invention further provides that in certain aspects the betalain is betalamic acid. In certain embodiments of the invention, the starting material is DOPA. In certain other embodiments of the invention, the organic material is L-DOPA. In certain embodiments, the one or more enzymes resulting in the transformation of organic material to betalamic acid is an engineered enzyme. In certain embodiments of the invention, 3,4-dihydroxyphenylalanine 4,5-dioxygenase (DODA) transforms DOPA or L-DOPA to betalamic acid. In certain embodiments, DODA could be an engineered enzyme. In certain embodiments of the invention, transformation proceeds through one or more chemical intermediates and the one or more chemical intermediates. In certain embodiments of the invention, one or more chemical intermediates comprises 4-(L-alanin-3-yl)-2-hydroxy-cis,cis-muconate 6-semialdehyde (4,5-seco-DOPA). In certain embodiments of the invention, betalamic acid is further reacted with one or more amino acids and/or amines to prepare one or more pigments. In certain embodiments of the invention, the one or more pigments are colors for addition to food. In certain embodiments of the invention, the one or more pigments are colors for cosmetics. In certain embodiments of the invention, the one or more pigments are ingredients in nutraceuticals. In certain embodiments of the invention, the one or more pigments is betanin, betanidin, betacyanin, betaxanthin, betalain, or a combination thereof. In another embodiment, the enzyme resulting in transformation of the organic material to a betalain is immobilized on a surface. In another embodiment, the enzyme resulting in transformation of the organic material to a betalain is not immobilized.

In other aspects of the invention, a composition for cell-free production of a betalain is provided. In such aspects of the invention, the composition comprises an enzyme that results in transformation of an organic material to a betalain. In certain aspects, the invention further provides that the betalain is betalamic acid. In certain embodiments of the invention, the organic material is DOPA. In certain embodiments of the invention, the organic material is L-DOPA. In certain embodiments, the one or more enzymes resulting in the transformation of organic material to betalamic acid is an engineered enzyme. In certain embodiments of the invention, 3,4-dihydroxyphenylalanine 4,5-dioxygenase (DODA) transforms DOPA or L-DOPA to betalamic acid. In certain embodiments, DODA could be an engineered enzyme. In certain embodiments of the invention, transformation proceeds through one or more chemical intermediates and the one or more chemical intermediates. In certain embodiments of the invention, one or more chemical intermediates comprises 4-(L-alanin-3-yl)-2-hydroxy-cis,cis-muconate 6-semialdehyde (4,5-seco-DOPA). In certain embodiments of the invention, betalamic acid is further reacted with one or more amino acids and/or amines to prepare one or more pigments. In certain embodiments of the invention, the one or more pigments are colors for addition to food. In certain embodiments of the invention, the one or more pigments are colors for cosmetics. In certain embodiments of the invention, the one or more pigments are ingredients in nutraceuticals. In certain embodiments of the invention, the one or more pigments is betanin, betanidin, betacyanin, betaxanthin, betalain, or a combination thereof. In another embodiment, the enzyme resulting in transformation of the organic material to a betalain is immobilized on a surface. In another embodiment, the enzyme resulting in transformation of the organic material to a betalain is not immobilized.

In some embodiments, the isolated betalain has a purity of about 10%, or about 20%, or about 30%, or about 40%, or about 50%, or about 60%, or about 70%, or about 80%, or about 90%, or about 95%, or about 99%, or about 100%.

In other embodiments, the isolated betalain has a purity of from about 10% to 95%, or from about 10% to 90%, or from about 10% to 80% or from about 10% to 70%, or from about 10% to 60%, or from about 10% to 50%, or from about 10% to 40%, or from about 20% to 95%, or from about 20% to 90%, or from about 20% to 80% or from about 20% to 70%, or from about 20% to 60%, or from about 20% to 50%, or from about 20% to 40%, or from about 50% to 95%, or from about 50% to 90%, or from about 50% to 80% or from about 50% to 70%, or from about 50% to 60%.

In some embodiments, the isolated betalamic acid has a purity of about 10%, or about 20%, or about 30%, or about 40%, or about 50%, or about 60%, or about 70%, or about 80%, or about 90%, or about 95%, or about 99%, or about 100%.

In other embodiments, the isolated betalamic acid has a purity of from about 10% to 95%, or from about 10% to 90%, or from about 10% to 80% or from about 10% to 70%, or from about 10% to 60%, or from about 10% to 50%, or from about 10% to 40%, or from about 20% to 95%, or from about 20% to 90%, or from about 20% to 80% or from about 20% to 70%, or from about 20% to 60%, or from about 20% to 50%, or from about 20% to 40%, or from about 50% to 95%, or from about 50% to 90%, or from about 50% to 80% or from about 50% to 70%, or from about 50% to 60%.

In some embodiments, the enzymes may be immobilized. In some embodiments, immobilized enzymes may be immobilized onto solid supports. Non-limiting examples of solid supports may include (but are not limited to) epoxy methacrylate, carboxymethyl-cellulose, starch, collagen, ion exchange resins, amino C₆ methacrylate, or microporous polymethacrylate. In further embodiments, various surface chemistries may be used for linking the immobilized enzyme to a solid surface, including but not limited to covalent, adsorption, ionic, affinity, encapsulation, or entrapment. In other embodiments, immobilized enzymes may be immobilized in crosslinked enzyme aggregates. In other embodiments, the enzymes are non-immobilized. Either immobilized or non-immobilized enzymes may be employed in batch or continuous synthesis. For example, an immobilized enzyme on a solid support may be used in a cartridge through which a reaction mixture passes, whereby an immobilized enzyme may catalyze modification of substrate to produce the product at a high titer. Alternatively, a continuous method may comprise micro mixing of enzyme solution and substrate to produce the product at a high titer, while continuously removing product, removing (e.g., recovering) substrate, or both. In some embodiments removed (e.g., recovered) substrate may be recycled to increase process efficiency and overall yield. In some embodiments, DODA enzymes are non-immobilized. In some embodiments, enzyme is recycled by ultrafiltration. In some embodiments ion exchange resins may be used to capture betalamic acid during production. For example, amine-functionalized solid support may be added to capture betalamic acid for continuous purification from reaction mixture.

In some embodiments, the enzymes used in the methods described herein are wild type enzymes from various bacterial, fungal, and plant species. In other embodiments, the enzymes used in the methods described herein are variants of the wild type enzymes.

In some embodiments, the temperature of the reaction may range from about 0° C. to about 80° C. In some embodiments, the temperature of the reaction may be about 10° C., e.g., about 0° C. to about 20° C., about 20° C. to about 30° C., about 30° C. to about 40° C., about 40° C. to about 50° C., about 50° C. to about 60° C., about 60° C. to about 70° C., or about 70° C. to about 80° C. In each case of ° C., “about” indicates ±1° C.

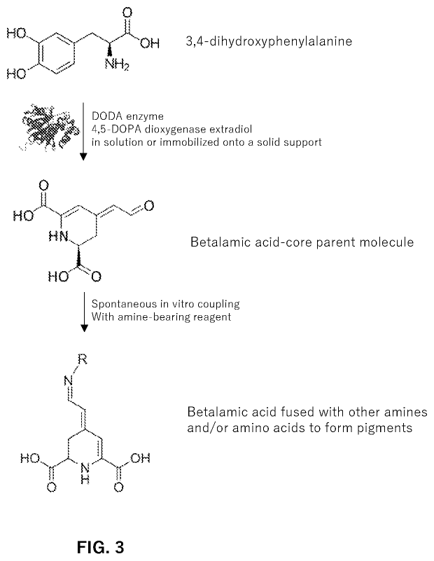

As shown in FIG. 3, 4,5-DOPA dioxygenase extradiol (DODA) is a key enzyme of the betalain biosynthetic pathway that opens the cyclic ring of DOPA or L-DOPA between carbons 4 and 5, thus producing an unstable seco-DOPA that rearranges nonenzymatically to betalamic acid. The isolation of DODA from plant and fungus has been previously described. See generally, Chrisinet et al., Plant Physiol. 2004 January; 134(1): 265-274; Bean et al., New Phytologist, 219(1), 287-296. The current invention may involve the use of any previously identified DODA enzyme from any species. The current invention preferably involves the use of an engineered variant of DODA.

As shown in FIG. 1, DOPA is a critical component of betalains biosynthetic pathway. A preferred stereoisomer of DOPA is L-DOPA. L-DOPA synthesis involves the 3-hydroxylation of L-tyrosine, cyclo-DOPA formation is via dopaquinone from the oxidation of L-DOPA. The invention provides that L-DOPA used in the invention is either sourced commercially or is also produced enzymatically in the cells or in a cell-free environment. Biosynthetic pathways for enzymatic production of L-DOPA are previously described. See generally, Schwinn K. E., New Phytol., 2016, DOI: 10.1111/nph.13901; Polturak et al., New Phytol., 2016, 210: 269-283.

Betalamic acid (BA) is the central chromophore of betalain pigments. The pigments are produced from spontaneous condensation of BA with amino acids or amines. The condensation of betalamic acid with amines and/or amino acids results in formation of betaxanthins. The type of amines and/or amino acids used for these reactions determines which betaxanthin is formed and the properties of said betaxanthin. Betalamic acid upon condensation with DOPA results in the formation of dopaxanthin. The condensation of betalamic acid with indoline derivatives results in formation of betacyanins.

The invention also provides for the use of pigments obtained from betalamic acid and/or betalains as food coloring, cosmetic pigments, other dyes, and nutraceuticals. In one embodiment, the pigments are betaxanthins, betacyanins, and dopaxanthin. In one embodiment, betacyanins produce a color palette including red, violet, purple, blue, orange, and yellow. In one embodiment, betaxanthins produce a color palette including red, violet, purple, blue, orange, and yellow. In one embodiment, a combination of betacyanin and betaxanthin produce colors including red, violet, purple, blue, green, orange, and yellow.

In one embodiment, the methods of the present invention produce one or more betaxanthins. In one embodiment, the betaxanthin comprises Vulgaxanthin I (glutamine-betaxanthin). In another embodiment, the betaxanthin comprises Indicaxanthin (proline-betaxanthin). In another embodiment, the betaxanthin comprises histamine-betaxanthin. In another embodiment, the betaxanthin comprises alanine-betaxanthin. In another embodiment, the betaxanthin comprises dopamine-betaxanthin. In another embodiment, the betaxanthin comprises valine-betaxanthin.

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for cell-free production of betalamic acid, wherein the method comprises: providing one or more enzymes in a cell-free medium, wherein the one or more enzymes result in transformation of an organic material to a betalain.

2. A method of claim 1, wherein the betalain is betalamic acid.

3. The method of claim 1, wherein the organic material is any of the stereoisomers of DOPA, or racemic DOPA mixtures thereof.

4. The method of claim 1, wherein the one or more enzyme is an engineered enzyme.

5. The method of claim 1, wherein the one or more enzyme is 3,4-dihydroxyphenylalanine 4,5-dioxygenase (DODA).

6. The method of claim 4, wherein the engineered enzyme is 3,4-dihydroxyphenylalanine 4,5-dioxygenase (DODA).

7. The method of claim 1, wherein the transformation proceeds through one or more chemical intermediates and the one or more chemical intermediates comprises any stereoisomer of 6-amino-4-[2-hydroxyethenyl]-2-oxohept-3-enedioic acid (4,5-seco-DOPA).

8. The method of claim 2, wherein betalamic acid is further reacted with one or more amino acids and/or amines to prepare one or more pigments.

9. (canceled)

10. (canceled)

11. (canceled)

12. (canceled)

13. The method of claim 8, wherein the pigment is a betalain.

14. The method of claim 8, wherein the pigment is selected from a group consisting of betanin, betanidin, betacyanin, betaxanthin, betalain, or a combination thereof.

15. (canceled)

16. (canceled)

17. A composition for cell-free production of a betalain, wherein the composition comprises: one or more enzymes in a cell-free medium, wherein the one or more enzymes result in transformation of an organic material to a betalain.

18. The composition of claim 17, wherein the betalain is betalamic acid.

19. The composition of claim 17, wherein the organic material is any stereoisomer of 3,4-dihydroxyphenylalanine (DOPA).

20. The composition of claim 17, wherein the one or more enzyme is an engineered enzyme.

21. The composition of claim 17, wherein the one or more enzyme is 3,4-dihydroxyphenylalanine 4,5-dioxygenase (DODA) from any number of plant, fungal, or bacterial species.

22. The composition of claim 20, wherein the engineered enzyme is 3,4-dihydroxyphenylalanine 4,5-dioxygenase (DODA).

23. The composition of claim 17, wherein the transformation proceeds through one or more chemical intermediates and the one or more chemical intermediates comprises any stereoisomer of 4-(alanin-3-yl)-2-hydroxy-cis,cis-muconate 6-semialdehyde (4,5-seco-DOPA), or a racemic mixture thereof.

24. The composition of claim 18, wherein betalamic acid is further reacted with one or more amino acids and/or amines to prepare one or more pigments.

25. (canceled)

26. (canceled)

27. (canceled)

28. (canceled)

29. The composition of claim 24, wherein the pigment is a betalain.

30. The composition of claim 24, wherein the pigment is selected from a group consisting of betanin, betanidin, betacyanin, betaxanthin, betalain, or a combination thereof.

31. (canceled)

32. (canceled)
